(19) United States
(12) Patent Application Publication
(10) Pub. No.: US 2022/0193285 A1
(43) Pub. Date: Jun. 23, 2022

(54) ARTICLE SANITIZING DEVICE AND SYSTEM

(71) Applicant: PCL PURE Products LLC, Denver, CO (US)

(72) Inventors: Chad Keuler, Winnipeg (CA); Sean Barnes, East St Paul (CA)

(21) Appl. No.: 17/644,964

(22) Filed: Dec. 17, 2021

Related U.S. Application Data
(60) Provisional application No. 63/199,320, filed on Dec. 18, 2020.

(51) Int. Cl. A61L 2/10

(52) U.S. Cl. CPC A61L 2/10; A61L 2202/122; A61L 2202/14; A61L 2202/11

(57) ABSTRACT

A sanitizing device for de-activating one or more pathogens located on an article. The sanitizing device includes a control unit, a container having an opening and a defined interior space for receiving a selected article. The sanitizing device includes at least one UV light source located within the interior space and a sensor unit located within the interior space in communication with the control unit. The sanitizing device receives the selected article within the interior space, the sensor detects at least one operating condition and communicates the operating condition to the control unit. The control unit selectively powers the UV light source to emit UV light from the UV light source toward the selected article based upon the operating condition.

### CROSS-REFERENCE TO RELATED APPLICATIONS

This application claims the benefit of U.S. Provisional Patent Application No. 63/199,320, filed Dec. 18, 2020, the entire disclosure of which is hereby incorporated by reference.

### BACKGROUND OF THE INVENTION

Increasing health safety concerns, such as those brought on by the recent COVID-19 pandemic, have increased the need for proper sanitization of objects and articles that commonly handled by several individuals and/or that may have potentially harmful pathogens and similar matter located thereon. For example, shopping carts, shopping baskets, and similar items that are frequently used by several individuals may have on the surfaces thereof, pathogens that may be harmful to future users of such items. Similarly, packages, luggage and other items are often handled by several individuals, which may transmit harmful pathogens onto the surfaces of such items. As a result, there is an increased need to provide for frequent cleaning and sanitization of such objects and articles in order to prevent subsequent handlers of such items from coming into contact with any potentially harmful pathogens located thereon.

Current solutions for sanitizing frequently handled articles or articles used by multiple persons in a short period of time, such as shopping carts, mobility scooters, and furniture in short-term lodgings, involve intensive manual labor and time-consuming effort. For example, shopping carts are often manually cleaned with disinfectant chemicals and liquids by wiping down each article after use. However, this cleaning solution is slow and cannot be employed to effectively sanitize large numbers of articles and degrades the surfaces of items being cleaned due to the chemicals being applied. Other solutions involve comprehensive systems that are used to sanitize articles by applying heat or disinfecting liquids or steam to articles processed through a cleaning chamber system. However, such systems are expensive to maintain and require significant space and oversight to effectively use. Further, such solutions are not adapted to a variety of use cases and are not suited for tracking of sanitized articles. Accordingly, a need exists for a solution that enables the sanitization of a variety of articles in a safe and effective manner.

### SUMMARY OF THE INVENTION

The present invention is directed toward a sanitizing device for de-activating one or more pathogens located on an article. The sanitation device may include a control unit, a container having an opening and defining an interior space for receiving a selected article, at least one UV light source located within the interior space, and a sensor unit located within the interior space in communication with the control unit. Further, the sanitation device receives the selected article within the interior space and the sensor may detect at least one operating condition and communicates the operating condition to the control unit, and the control unit may selectively power the UV light source to emit UV light from the UV light source toward the selected article based upon the operating condition. The UV light source may be a 222 nanometer wavelength UV light lamp. In another embodiment, the interior space may be defined by at least two panels and a reflective material may be disposed on an interior surface of each of the at least two panels. In one embodiment, the selected article may be a shopping cart.

In one embodiment, the sensor may be a camera unit that scans a QR code located on the selected article when the selected article is inserted into the device. In another embodiment, the control unit powers the at least one UV light source only after scanning the QR code.

In one embodiment, the container may be the cargo box of a vehicle, such as a cargo truck or the trailer of a tractor trailer, and the sanitation device may be portable for delivery and or rental services wherein a rented item can be sanitized before and/or after delivery to a new customer.

In another embodiment, the present invention is a room sanitizing device for de-activating one or more pathogens located in the air or on an article within a room. The room sanitizing device may include a control unit, an interior space defined by a first sidewall, a second side wall, and third side wall and the interior space is sized to receive a selected article. The present room sanitizing device may also include at least one UV light source located within the interior space, the UV light source being a UV light bulb mounted in a ceiling fixture, and a sensor unit located within the interior space in communication with the control unit. In one embodiment, the sensor unit detects either an operating condition or a safety off condition, wherein the operating condition occurs when the sensor unit does not detect a person in the room, and the control unit then selectively powers the UV light source to emit UV light from the UV light source toward the selected article based upon the operating condition. In one embodiment, the control unit operates the UV light source for a certain period of time based upon the distance and wattage of the UV light from the UV light source. In one embodiment, the sensor unit may be a thermal sensor for detecting a person and, alternatively, the sensor unit may be a motion detector, camera, or switch disposed on a door to the room. The room sanitation device may also include an embodiment wherein the at least one UV light source may be portable.

Another embodiment of the present invention is directed toward a sanitizing device for de-activating one or more pathogens. In one embodiment, the device is directed toward sanitizing wheeled articles. The sanitizing device may include a control unit; a container having an opening and defining an interior space for receiving the wheeled article; a plurality of UV light sources located within the interior space; and a sensor unit located within the interior space in communication with the control unit. In one embodiment, the device receives the wheeled article within the interior space, and the sensor detects at least one operating condition and communicates the operating condition to the control unit, and the control unit selectively powers the plurality of UV light sources to emit UV light from the UV light source toward the wheeled article for a defined period of time based upon the at least one operating condition.

In one embodiment, the interior space may be defined by at least two side panels and a reflective material or reflector panels may be disposed on an interior surface of each of the at least two side panels.

Other aspects and advantages of the present invention will be apparent from the following detailed description of the preferred embodiments and the accompanying drawing figures.

### DESCRIPTION OF THE SEVERAL VIEWS OF THE DRAWINGS

In the accompanying drawings, which form a part of the specification and are to be read in conjunction therewith in which like reference numerals are used to indicate like or similar parts in the various views:

FIG. 1 is a perspective view of a sanitization device in accordance with one embodiment of the present invention;

FIG. 2 is a front elevation view of the sanitization device of FIG. 1 cut along the line 2-2;

FIG. 3 is a side sectional elevation view of the sanitization device of FIG. 1, cut along the line 3-3 illustrating a shopping cart inserted therein;

FIG. 4 is a side elevation view of the sanitization device of FIG. 1, illustrating a wheelchair inserted therein;

FIG. 5 is a side elevation view of the sanitization device of FIG. 1, illustrating shopping baskets inserted therein;

FIG. 6 is a schematic diagram of a sanitization device in accordance with one embodiment of the present invention;

FIG. 7 is a perspective view of a sanitization device in accordance with one embodiment of the present invention;

FIG. 8 is a top plan view of a sanitization device having an article inserted therein in accordance with one embodiment of the present invention;

FIG. 9 is a perspective view of a sanitization device in accordance with an alternative embodiment of the present invention;

FIG. 10A is an end schematic views of a sanitization device in accordance with an alternative embodiment of the present invention;

FIG. 10B is section top view of the sanitization device in accordance with FIG. 10A cut along the line 10-10;

FIG. 11A is a perspective schematic view of a sanitization device in accordance with an alternative embodiment of the present invention;

FIG. 11B is a schematic section end view of a sanitization device in accordance with the embodiment in FIG. 11A cut along the line 12-12;

FIG. 11C is a schematic section top view of a sanitization device in accordance with the embodiment in FIG. 11A cut along the line 13-13;

FIG. 12 is a perspective cut-away view of a sanitization device in accordance with an alternative embodiment of the present invention wherein the container is a box on a truck;

FIG. 13 is a perspective view of a sanitization device in accordance with an alternative embodiment of the present invention wherein the container is room in a building;

FIG. 14A is a perspective cut-away view of a sanitization device in accordance with an alternative embodiment of the present invention that is portable and has a flexible configuration;

FIG. 14B is a perspective cut-away view of a sanitization device in accordance with an alternative embodiment of the present invention that is portable and has a flexible configuration; and

FIG. 14C is a perspective cut-away view of a sanitization device in accordance with an alternative embodiment of the present invention that is portable and has a flexible configuration.

### DETAILED DESCRIPTION OF THE INVENTION

The invention will now be described with reference to the drawing figures, in which like reference numerals refer to like parts throughout. For purposes of clarity in illustrating the characteristics of the present invention, proportional relationships of the elements have not necessarily been maintained in the drawing figures.

The following detailed description of the invention references specific embodiments in which the invention can be practiced. The embodiments are intended to describe aspects of the invention in sufficient detail to enable those skilled in the art to practice the invention. Other embodiments can be utilized and changes can be made without departing from the scope of the present invention. It will be appreciated that some or all of the various features and structures described and shown with respect to each of the specific embodiments referenced herein may be combined to form additional or alternative embodiments having such combinations and that such combinations are within the scope of the present invention.

Referring to the figures, the present invention is directed toward a sanitizing device 10 for sanitizing selected articles (such as shopping carts, wheelchairs, packages, containers, cartons, luggage items and other objects) from potentially harmful viruses, bacteria, pathogens and other organic matter that may be present on the surface of the selected article. As described in greater detail herein, an object or article may be inserted into device 10 and exposed to a UV light that is designed to eliminate and/or de-activate potentially harmful pathogens that exist on the surfaces of the object or article. Once the article is inserted into device 10, device 10 can emit UV light for a selected period of time within the interior of device 10 in order to de-activate a sufficient amount of any pathogens present on the surfaces of the article. Once the sanitization period is completed, device 10 can stop emitting UV light and the article can be safely removed from device 10.

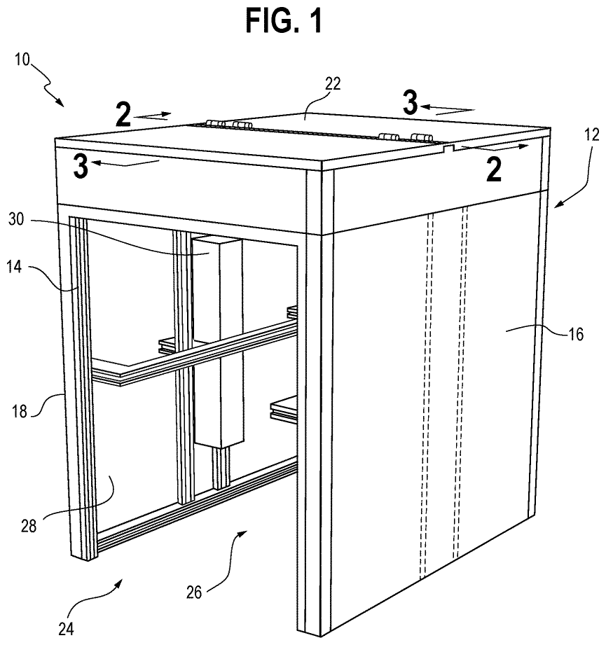

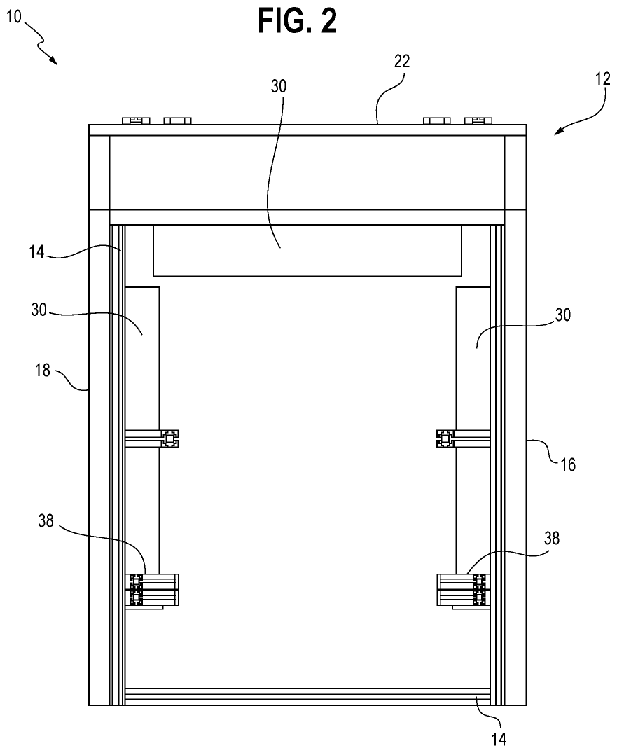

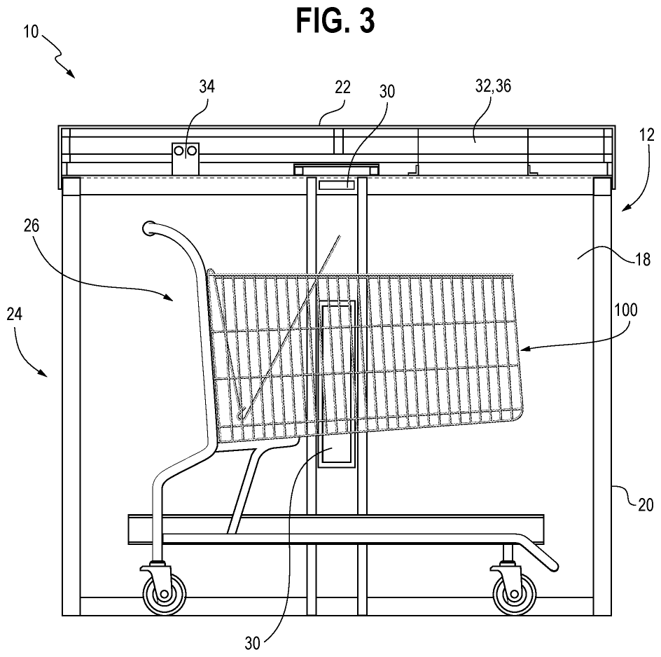

As shown in the several figures, device 10 can include a container-shaped structure 12 that is designed and configured to receive a selected article or object to be inserted therein for sanitization. As best shown FIGS. 1-5, container 12 can include an outer frame 14 that forms the shape and structure of container 12 and provides an interior sanitization space 26 within the interior of container 12. Container 12 can include a first sidewall panel 16, a second sidewall panel 18, and a rear wall panel 20, each secured around the exterior of frame 14 to enclose container 12. Container 12 can further include a cover or top panel 22 attached to the upper end of outer frame 14. Collectively, panels 16-22 can provide an opening 24 opposite rear wall panel 20 that can provide access to the interior 26 of container 12 and allow articles or objects to be placed into and removed from container 12. Outer frame 14 and panels 16-22 can be constructed from any suitable material or materials and can be comprised of individual components connected together or constructed as an integral structure to form container 12. Container 12 may additionally include one or more guide rails, hanging brackets or similar structures 38 located within interior space 26 and connected to frame 14 in order to assist in the proper positioning and placement of selected articles 100 within device 10 according to certain embodiments of the present invention.

As further shown in FIGS. 1-5, device 10 can include one or more ultraviolet (UV) light sources 30 positioned within the interior space 26 of container 12. UV light sources 30 can be secured to outer frame 14 and/or panels 16-22 of container 12 using any suitable methods or means. According to one embodiment, device 10 includes three UV light sources 30 attached along first sidewall panel 16, second sidewall panel 18 and top panel 22. According to certain embodiments, each UV light source 30 can comprise a 250 v light lamp that emits UV light at a 222 nanometer wavelength; however, it is recognized that any suitable number of UV light sources 30 and UV wavelengths may be used and positioned within the interior space 26 of container in any suitable arrangement for sanitizing items. One embodiment could use UVC light that is emitted toward selected articles placed into a compartment in order to destroy or de-activate any pathogens located on the surfaces of the articles. A preferred UV wavelength is 222 nm. One particular advantage of 222 nm UV light sources is that UV light at 222 nm has been shown to be considered generally safe for humans and does not damage or degrade materials, such as plastic, metal, etc., while still providing effective de-activation of pathogens. According to certain embodiments, the one or more UV light sources 30 can each comprise a 222 nm STERILRAY™ lamp and designed and configured in accordance with the technologies and disclosures set forth in U.S. Pat. Nos. 8,481,985, 8,753,575, 8,975,605, 9,700,642, CA Pat. No. 2,600,923 and CA Pat. No. 2,749,283, the entire disclosures of which are incorporated herein by reference.

The UV light sources 30 can be configured and positioned within the interior space 26 of container 12 in order to maximize the sanitization efficiency of device 10. As shown in the several figures, an individual UV light source 30 may be provided on each of first sidewall 16, second sidewall 18 and top wall 22 so that UV light emitted from each UV light source 30 is directed toward an article 100 that is placed within interior space 26 of container 12; however, it is recognized that any number of suitable positioning arrangements of UV light sources 30 may be used in various embodiments of the present invention. Additionally, according to certain embodiments, the interior surfaces of one or more of panels 16-22 can include a coating, fabric or other material 28 with reflective properties that can increase the effectiveness of UV light sources 30 by reflecting and redistributing UV light throughout the interior space 26 of container 12. The reflective material 28 may be configured as a sheet of fabric or material that is secured to the interior surfaces of container 12 or may be a coating material that is painted or otherwise applied to one or more of the interior surfaces of container 12. The reflective material 28 may also be configured as individual panels oriented within interior space 26 to direct emitted UV light toward the direction of the article 100 placed therein.

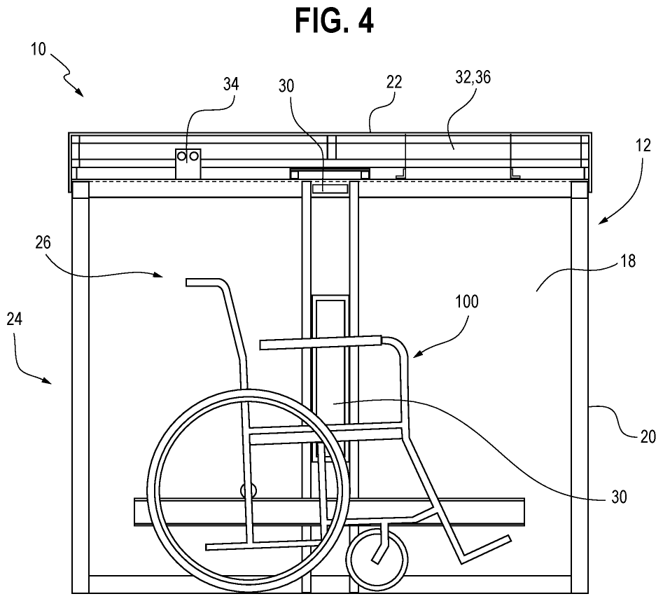

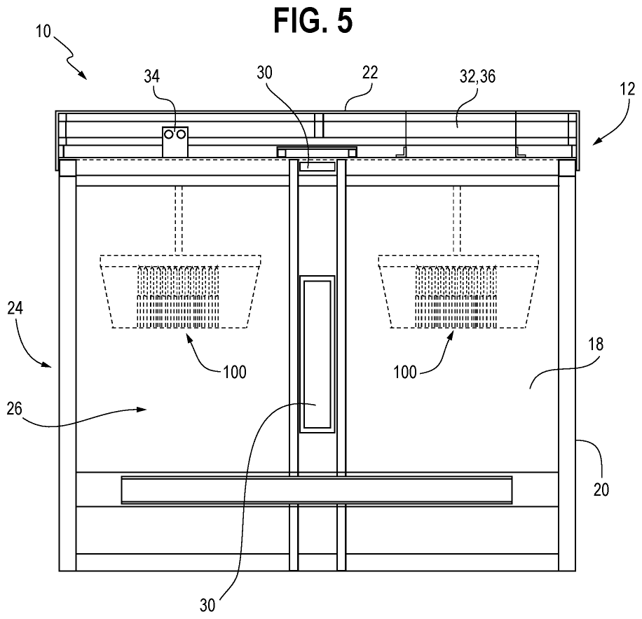

As best shown in FIGS. 4-5, device 10 can include a power unit 32 configured to supply power for the operation of UV light sources 30 and device 10 overall. According to one embodiment, power unit 32 may be provided in or on top panel 22 of container 12. Power unit 32 may be configured as any suitable means for supplying power to device 10, including without limitation, an internal battery unit and/or a power outlet and power control relay.

As also shown in FIGS. 4 and 5, device 10 can include a camera or scanner unit 34 that can be positioned within container 12 and configured to read and/or identify the object inserted into the interior space 26 of container 12. According to one embodiment, camera unit 34 can be configured to scan and identify a QR code located on the article 100 inserted into container 12 for sanitization or simply used to detect the presence and/or type of article 100 as a condition to allow the UV lights 30 to turn on for sanitation. In such an embodiment, camera unit 34 reads the QR code on the article 100 or the presence and/or type of article 100 and directs the activation of device 10 if the QR code meets certain specified criteria or an article is deemed present. Thus, this acts as a safety device so that the device 10 only operates when an object is present inside the device. The noted camera 34 may also be used as a safety device in some embodiments, so that camera 34 will recognize humans within the container and immediately stop the sanitization process. According to other embodiments, camera unit 34 may instead be configured as an RFID scanner or any suitable technology used to identify certain articles 100 located within container 12. Camera unit 34 may be connected to power unit 32 and a processor (not shown) in order to power and operate camera unit 34 within device 10.

As also shown in FIGS. 4-5, device 10 can include a control unit 36 that can be configured to control the operation of device 10 (including UV light sources 30 and camera unit 34). Control unit 36 can be configured as any suitable computer operating component with programming to enable automated and/or manual operation of device 10 and container 12 as described in greater detail below. Control unit 36 may be connected to power unit 32, UV light sources 30, and/or camera unit 34 to control the operation thereof.

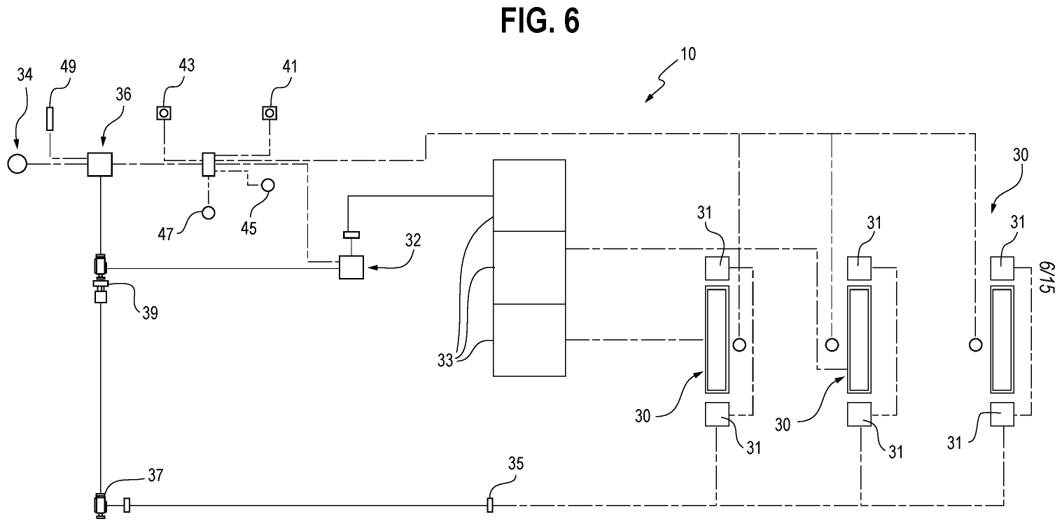

FIG. 6 best illustrates the schematic electrical controls and element of one embodiment of the device 10 of the present invention. As shown, the schematic shows how the control unit 36 is connected to the power unit 32 and UV light sources 30. Namely, control unit 32 is operably connected to a power supply 33 for each light fixture 30. In this embodiment, a three-volt power supply is utilized though other voltages are within the scope of the present invention. Each light fixture 30 may include one or more cooling fans 31 to cool the fixtures to prevent overheating.

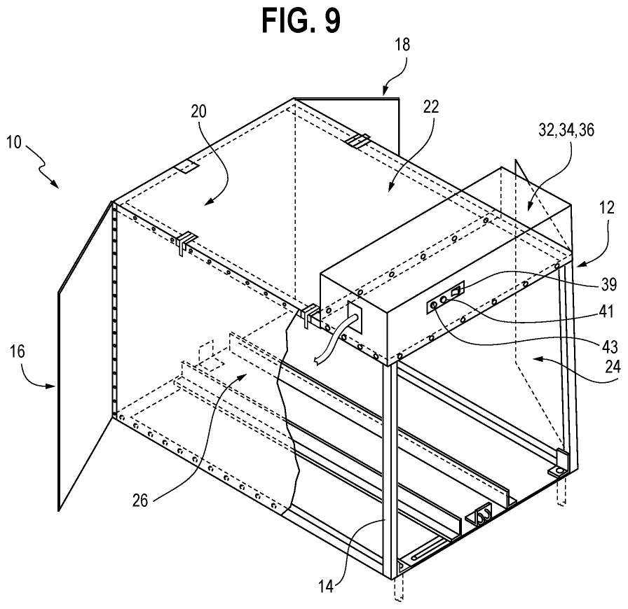

As further shown in FIG. 6 and FIG. 9, camera 34 is also connected to control unit 36. The configuration of device 10 may also include a motion sensor 37 configured to sense the present of a person and their movement within the container as a safety precaution. Power may be distributed through a junction 39 which diverts power to both control unit 36 and power unit 32 and power supplies 33. In general, a sensor like camera 34 or motion sensor 37 is disposed to determine either a positive condition within container 26 (the right item is in the container 26) thereby allowing the device 10 to operate, or a negative condition within container 26 (the wrong item is in the container 26) thereby instructing control unit 36 to deactivate the UV light source 30 from operating. Further, control unit 36 may include an external by-pass button 41, a safe reset button 43, a buzzer 45, and a status indicator 47. The status indicator 47 may be a red/green light or other indicator which provides the user a status of the operation of the device 10. Buzzer 45 may be a piezo buzzer and may be used to indicate completion of the sanitation or other quality of the operation of the device 10. In some embodiments, the control unit 36 may have having wireless communication access through a cellular antenna 49 or other wired or wireless communication method.

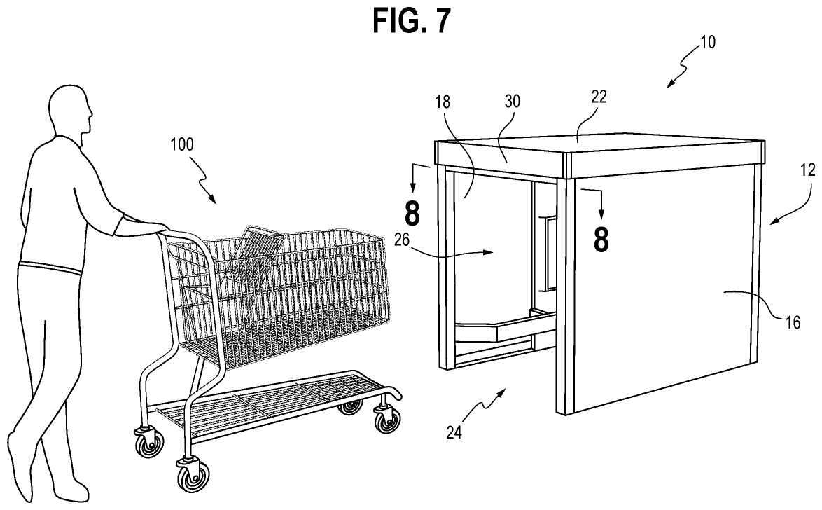

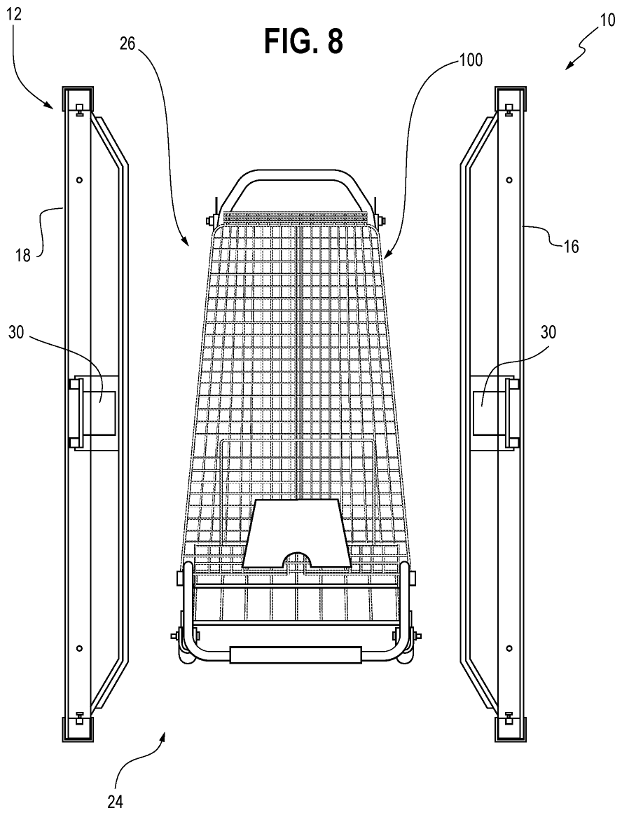

FIGS. 7-9 illustrate the operation of device 10 according to selected embodiments of the present invention. As shown, an article 100 (such as a shopping cart, wheelchair, shopping basket, carton, package, or similar item) that is desired to be sanitized can be inserted into container 12 through opening 24 and positioned within the interior space 26 of container 12. Once inserted, the camera unit 34 can scan a QR code (or similar unique identifier technology such as RFID) provided on the article 100 or simply to determine the presence and/or type of an article 100, which is relayed to the control unit 36. Upon reading the QR code or determining the presence and/or type of the object, the control unit 36 can direct the operation of device 10 based on one or more parameters specific to the type of article 100. The control unit 36 can then signal the activation of the UV light sources 30 in order to emit UV light within the interior space 26 of container 12 at an intensity level and exposure time for the type of product. In addition, the device can be controlled using a simple on/off switch or button which operates the UV light sources for a manually or automatically determined period of time. The camera 34 or other sensor can be used as a safety feature to verify the presence of a desired item or not activate if an undesired item or person is in the sanitization chamber. The UV light from UV light sources 30 can contact the surfaces of the article 100 and de-activate a sufficient amount of any pathogens located on the surfaces of article 100. Once the sanitization period or duration of exposure is completed, control unit 36 can direct the de-activation of UV light sources 30 and/or buzzer 45 and the article 100 may be removed from the container 12.

According to certain embodiments, the QR code located on the article 100 can determine the specific operation of device 10 based on one or more parameters. For example, the QR code can indicate to device 10 the length of time the UV light sources 30 should be activated and/or the power level for the UV light sources 30 as necessary to effectively de-activate any potential pathogens located on the surfaces of the specific article 100. In certain embodiments, device 10 can be configured to achieve a minimum de-activation of 99.9% or log 3 reduction of pathogens located on the surfaces of article 100 based on the parameters associated with the identified QR code.

Device 10 can also be configured to only operate after camera unit 34 scans an approved QR code or detects the presence and/or type of the article within the container 12. This can enable device 10 to operate only when approved articles 100 are inserted into container 12 in order to improve the safety of device 10. Device 10, via control unit 36, can further be configured to collect and transmit additional information related to the operation of device 10. Such communication can occur through cellular antenna 49 or other known electronic communication method. For example, device 10 can be remotely monitored for product quality control, run time of the UV light sources 30, the amount of uses and inventory control and tracking of all scanned articles 100 used within device 10. Moreover, external bypass button 43 may be used to stop the operation of the lights if the fully cycle needed to be cut short by a user. Safe reset button 43 may be used to start a sanitation process over again and/or in some embodiments, reset button may be to reset control unit 36 if it is malfunctioning.

As illustrated in FIGS. 7-9, device 10 can be designed and configured for use with certain articles 100, such as shopping carts, wheelchairs, and shopping baskets, in order to effectively sanitize and de-activate potentially harmful pathogens, such objects that are subject to public use by many different individuals. Device 10 can additionally be configured and used in any number of different applications as illustrated in the various embodiment shown in the several figures. For example, device 10 can be adapted for use in connection with a conveyor belt to sanitize luggage, packages, food items or the like that travel along the conveyor belt. In such an application, container 12 may be positioned over a selected location on the conveyer belt and articles 100 (such as a piece of luggage or a package) can move along the conveyer belt and be automatically fed into container 12. Once positioned within container 12, the camera unit 34 may detect the presence and/or type of the article 100 or scan a QR code located on the article 100 and device 10 can activate UV light sources 30 to complete the sanitization process. Upon completion of the sanitization process, the conveyer belt can operate to transfer the article 100 beyond container 12 and a new article 100 can be positioned within container 12.

In another embodiment, the device 10 may be used to sanitize a number of smaller individual items at one time. In this embodiment, preconfigured trays or carts may be configured to hold a plurality of items in a spaced apart manner, these trays or carts can be placed into the device 10, the UV light sources 30 turned on to expose the surface of each item to the UV light. Such applications may apply to sporting equipment, utensils, instruments, tools, houseware items, or any other items.

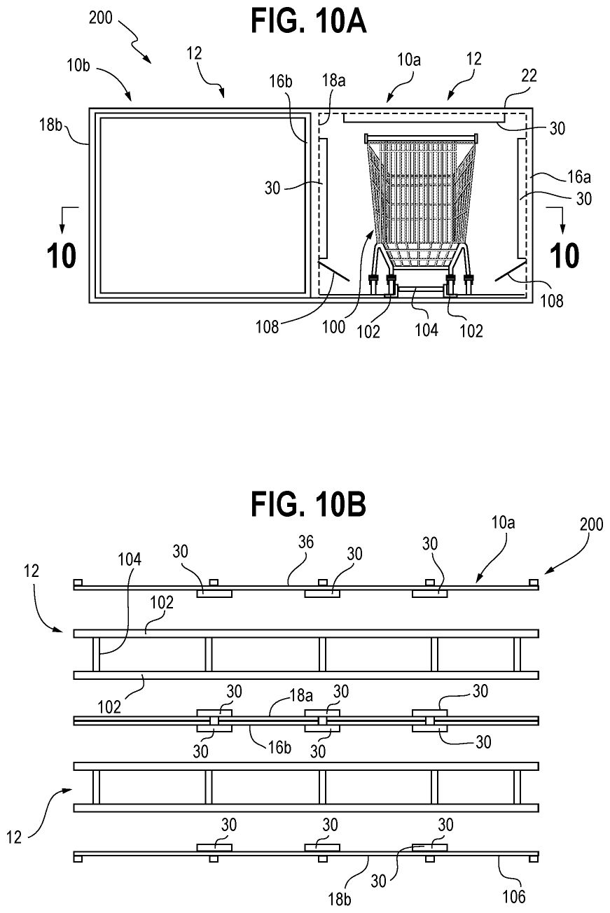

FIGS. 10A and 10B comprise an embodiment of the device 200 of the present invention which may be used to sanitize articles 100, such as a shopping cart, when they are in a stacked arrangement. As shown in FIG. 10A, broader device 200 a first device 10a and a second device 10b are arranged in a parallel arrangement. As shown in FIG. 10B, the length of top panel 22 and side walls 16a,b and 18a,b is increased to provide a length longer than article 100, which in one embodiment may allow a stack of shopping carts to be inserted into device 10a and 10b. Further, the number of UV light sources 30 may be increased and distributed along the length of top panel 22, and sides 16 and 18 at a distance to provide sufficient UV light exposure within the device 10. The embodiment shown includes three sets of UV light sources 30 distributed along the length of device 200

As shown in FIGS. 10A and 10B, in this embodiment, device 10a and 10b may include shopping cart guide 106, which comprises at least one guide rail 102. In one embodiment, there are two guide rails 102 separated by a plurality of spacer rods 104 which ban be mechanically adjusted to vary the spacing between rails 102. Guide rails 102 are shown as an angle shape, but could be any structural shape, including rectangular or circular tube shapes. Guide 106 helps to ensure that the shopping carts 100 are positioned within the device at an even spacing from the UV light sources 30 so as to provide the necessary disinfecting UV exposure. In addition, devices 10a and 10b may include a UV light reflector 108 located near the bottom of the sidewalls 16 and 18 so as to reflect UV light from below the shopping cart 100 to improve the distribution and coverage of the disinfecting UV light. Guides 102 may be arranged at any spacing or arrangement to correspond to the type of article 100 being sanitized.

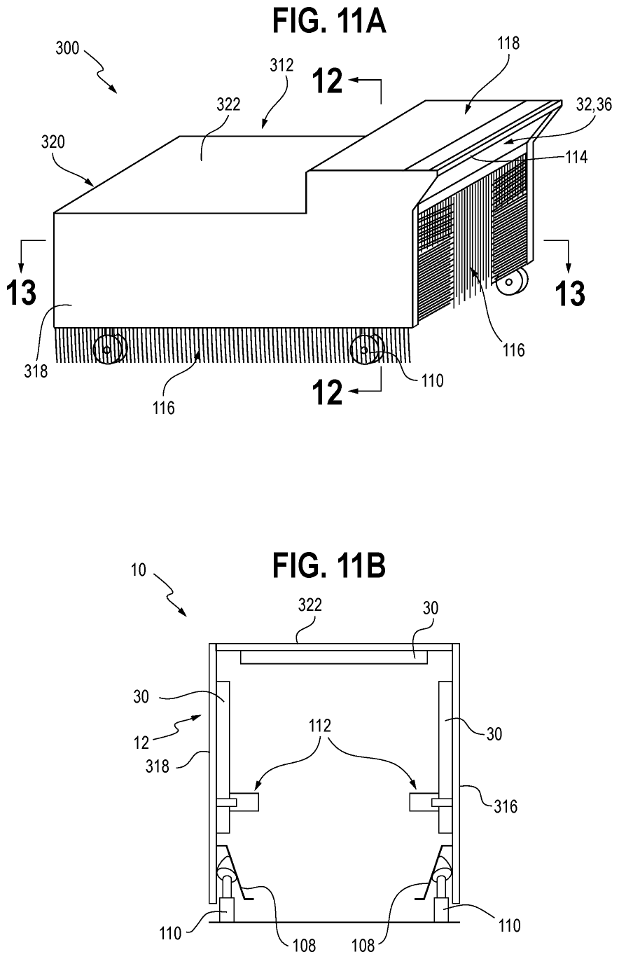

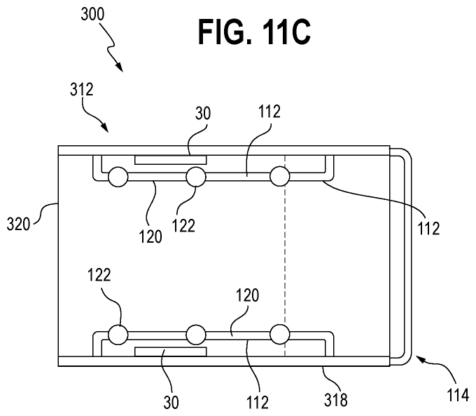

FIGS. 11A, 11B and 11C illustrate sanitizing device 300, which is another embodiment of the sanitizing device of the present invention. In FIG. 11A, device 300 is shown having a container 312 defined, at least in part, by a first sidewall 316, a second sidewall 318, a rear wall 320, and a top panel 322. These walls and panels are mounted on a frame (not shown, but similar to as described above). Sanitizing device 300 is portable and is mounted on wheels 110 located near each corner, front and back. In one embodiment, wheels 110 may be adjustable in order for a user to adjust the height of the top panel 322 to accommodate articles of various heights. Further, device 300 may include an arrangement of brush barrier 116 on the front side and on the bottom (near the bearing surface) of sides 316 and 318 and rear wall 320. In one embodiment, device 300 may be pulled using handle 114 (see FIG. 11A) over an item for sanitization or an article may be inserted therein. Further, device 300 may include a battery power source 118 and power unit 32 and control unit 36 disposed near the handle 114. As may be appreciated by a person of skill in the art, battery 118 may be rechargeable and selected to provide a certain length of sanitizing light exposure.

Sidewalls 316 and 318 may also include a roller guide bar 112 disposed in a middle portion of the sidewalls. As shown in FIG. 11C, each roller guide bar 112 may comprise a bar 120 having a plurality of rollers 122 mounted for rotation there on. These roller guide bars 112 both guide the device 300 over the article when pulled through, prevent an article from hitting the UV light sources 30 disposed on the respective sidewalls 316 and 318. As shown in FIG. 11B, device 300 may also include a reflector 108 which extends at least partially along the lengths of sidewalls 316 and 318. In one embodiment the reflector 108 does not protrude into the container 14 more than roller guide bars 122 so as to prevent the reflector 108 from being damaged by the article or reduce the instance of such damage.

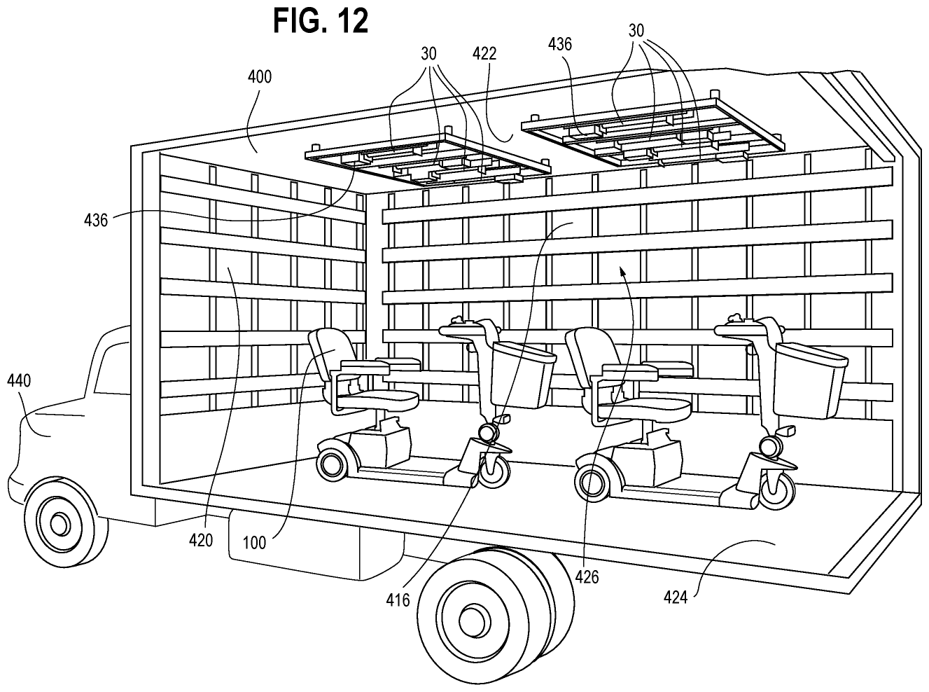

FIG. 12 illustrates sanitizing device 400, which is another embodiment of the sanitizing device of the present invention. Sanitizing device 400 is all or a portion of the cargo box of a vehicle 440, wherein the vehicle is a motor vehicle like a truck, however a similar configuration could be implemented in an enclosed cargo trailer, such as a semi-trailer or other cargo trailer. As shown in FIG. 12, sanitizing device 400 may include a first side wall 416, a second sidewall (not shown, but similar to the prior embodiments), a rear wall 420, top panel 422, and floor 424. There is also typically a door (not shown) of the device 400 as known in the art to enclose and secure the interior 424 of the device 400. Two clusters of UV light sources 30 are mounted to the top panel 422; however, any number of clusters or any pattern of UV light sources 30 may be arranged on any combination of the first sidewall 416, second sidewall, rear wall 420 and top panel 422 to provide the necessary UV light exposure to sanitize articles 100. In this embodiment, articles 100 are an electric mobility scooter, but it could be any articles carried in the interior 426 of device 400. Further, control units 436 for each cluster of UV light sources 30 may be mounted in proximity to the UV light sources 30.

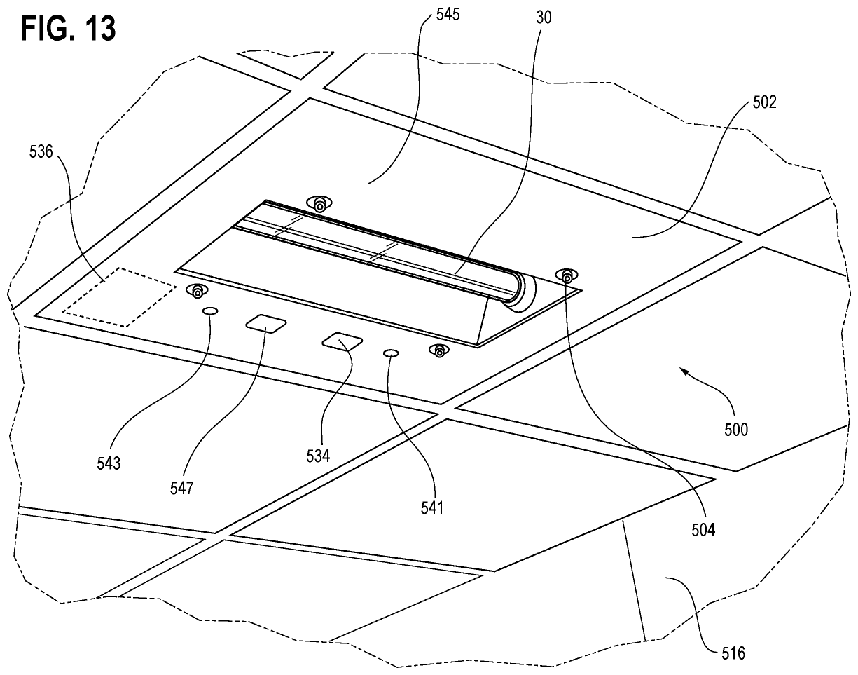

FIG. 13 illustrates sanitizing device 500, which is another embodiment of the sanitizing device of the present invention. Sanitizing device 500 is the interior of a room in a residential or office setting. As shown in FIG. 13, sanitizing device 500 may include a first side wall 516, a second sidewall (not shown, but similar to the prior embodiments), a rear wall (not shown, but similar to the prior embodiments), top panel 522, and floor (not shown, but similar to the prior embodiments). In one embodiment, there is also a front wall (not shown, but commonly encountered) to form an enclosed room in a building. There is also typically a door (not shown) of the device 500 as known in the art to enclose and secure the interior 524 of the device 400. UV light sources 30 are mounted to the top panel 422; however, any number of clusters or any pattern of UV light sources 30 may be arranged on any special arrangement in top panel 522, or in any combination of the first sidewall 516, second sidewall, rear wall and top panel 522 to provide the necessary UV light exposure to sanitize articles 100. In one embodiment, the UV light sources 30 are UV light bulbs mounted in a ceiling fixture that is mounted to the ceiling (the top panel 422). In this embodiment, articles 100 are the air and elements in the interior 524 of the device 500. Further, control units 536 for each UV light sources 30 may be mounted in proximity to the UV light sources 30, generally behind a panel 545. Further, control unit 536 may include an external by-pass button 541, a safe reset button 543, and a status indicator 547 which operate as described above. Finally, device 500 may include a camera 534 and or a heat-sensor to determine whether a person is in the interior 524 of device 500 so that control unit 536 can activate the UV light sources 30 only when a person is not in the interior 524 of device 500 and, in some embodiments, will only trigger operation after the sensor unit has detected that a person has been present in device 500 or the room.

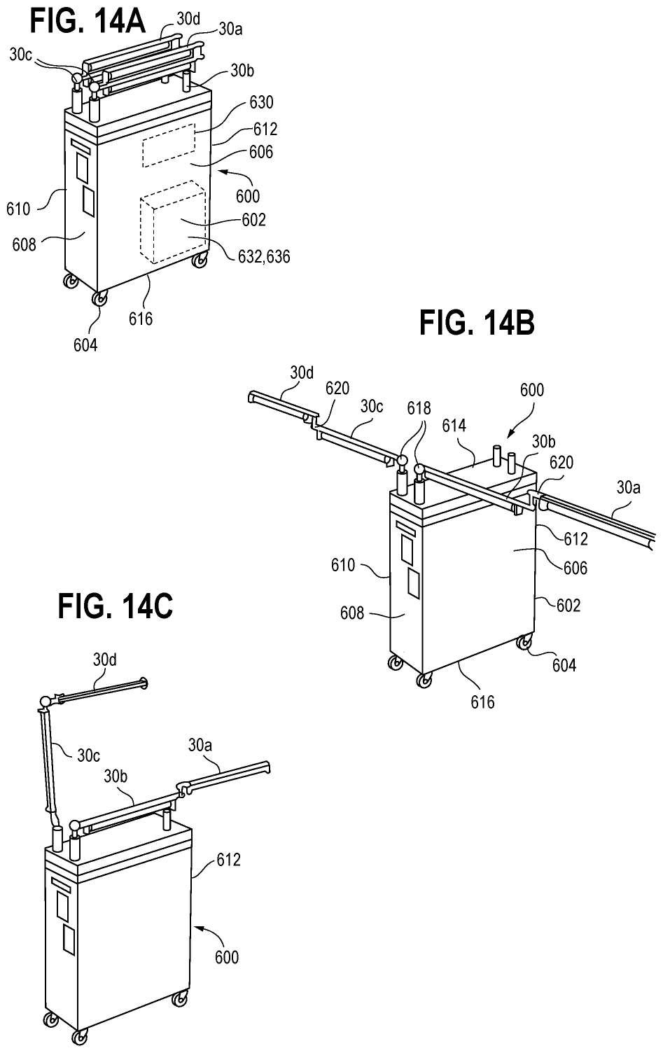

FIGS. 14A-C illustrates an embodiment of an UV light unit 600, wherein UV light unit 600 comprises a container 602 on four wheels 604. Container 602 may be a box having a first side 606, a second side 610, a front side 608, and a back side 612. Container 602 may also include a top 614 and a bottom 616. Container 602 may house a battery 630, a power unit 632, and a control unit 636 which operate as described above. Alternatively, the power source may be a plug in to conventional wired electric supply common in residential and commercial environments. UV light unit 600 is arranged to be portable and moveable from room to room.

FIGS. 14A-C illustrate the UV light sources 30a, 30b, 30c, and 30d, which are arranged to be moveable and arrangeable in a number of configurations. As shown in FIG. 14A, UV light sources 30a, 30b, 30c, and 30d are arranged in a folded position for transport. As shown in FIG. 14B, UV light sources 30a, 30b, 30c, and 30d are arranged in a laterally extended position, wherein UV light sources 30a and 30b are arranged as an articulating arm and extended in a direction perpendicular to first side 606 and away from first side 606. Similarly, UV light sources 30c and 30d are arranged as an articulating arm and extended in a direction perpendicular to second side 610 and away from second side 610. UV light sources 30b and 30c are connected to container 602 with an articulating joint, wherein a position and orientation of UV light sources 30b and 30c can be arranged in any direction both a 360-degree rotation about a vertical axis and at least a 180-degree rotation about a horizontal axis (vertical axis arranged parallel to a line normal to the plane defined by top 614.

As shown in FIG. 14c, UV light sources 30a, 30b, 30c, and 30d can be positioned in various arrangement, for example, UV light sources 30a and 30b extending in a linear manner in a direction away from back side 612 in a horizontal configuration, UV light source 30c is arranged in a substantially vertical orientation, with UV light source 30d being arranged in a horizontal configuration at a position higher from a ground surface than UV light sources 30a and 30b. As can be appreciated, the flexibility in the positioning of UV light sources 30a, 30b, 30c, and 30d allows a user to position the UV light sources in rooms to allow for apply UV light to items or fixtures in the room.

In use, UV light unit 600 may be wheeled into any area which is desired to be sanitized, such as a hotel/motel room, an airplane, a cruise ship, a suite in a sports arena, an office or conference center, virtually anywhere. Once at a desired location, UV light sources 30a, 30b, 30c, and 30d can be arranged and deployed to ensure light is projected toward the desired surfaces. Because of the articulating joint, the position of the UV light sources 30a, 30b, 30c, and 30d can be arranged in numerous configurations as shown. An operator can turn the machine on and a sensor can sense once the operator has left the room and then operate the UV light sources 30a, 30b, 30c, and 30d for the required amount of time.

From the foregoing, it will be seen that this invention is one well adapted to attain all the ends and objects hereinabove set forth together with other advantages which are obvious and which are inherent to the structure. It will be understood that certain features and sub combinations are of utility and may be employed without reference to other features and sub combinations. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. Thus, there has been shown and described several embodiments of a novel invention. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “may include” and not as “required”. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention.

What is claimed is:

1. A sanitizing device for de-activating one or more pathogens located on an article, said sanitizing device comprising:
a control unit;
a container having an opening and defining an interior space for receiving a selected article;
at least one UV light source located within said interior space; and
a sensor unit located within said interior space in communication with the control unit;
wherein said sanitation device receives said selected article within said interior space, said sensor detects at least one operating condition and communicates said operating condition to said control unit, and said control unit selectively powers said UV light source to emit UV light from said UV light source toward said selected article based upon said operating condition.

2. The device of claim 1, wherein said sensor is a camera unit that scans a QR code located on said selected article when said selected article is inserted into said device.

3. The device of claim 2, wherein said control unit powers said at least one UV light source only after scanning said QR code.

4. The device of claim 1, wherein said interior space is defined by at least two panels and wherein a reflective material is disposed on an interior surface of each of said at least two panels.

5. The device of claim 1, wherein said at least one UV light source comprises a 222 nanometer UV light lamp.

6. The device of claim 1, wherein said container is a room in a building, said UV light source is one or more ceiling mounted light fixtures, said sensor unit is a thermal sensor for detecting a person.

7. The device of claim 1, wherein said container is a cargo box of a vehicle.

8. The device of claim 1, wherein said selected article is a shopping cart.

9. A room sanitizing device for de-activating one or more pathogens located in the air or on an article, said room sanitizing device comprising:
a control unit;
an interior space defined by a first sidewall, a second side wall, and a third side wall for receiving a selected article;
at least one UV light source located within said interior space, the UV light source being a UV light bulb mounted in a ceiling fixture; and
a sensor unit located within said interior space in communication with the control unit;
wherein said sensor unit detects an operating condition or a safety off condition, wherein said operating condition occurs when said thermal sensor unit detects a person in the room, and said control unit selectively powers said UV light source to emit UV light from said UV light source toward said selected article based upon said operating condition.

10. The room sanitation device of claim 9, wherein said sensor unit is a thermal sensor for detecting a person.

11. The room sanitation device of claim 9, wherein the at least one UV light source is portable.

12. A sanitizing device for de-activating one or more pathogens located on a wheeled article, said device comprising:
a control unit;
a container having an opening and defining an interior space for receiving said wheeled article;
a plurality of UV light sources located within said interior space; and
a sensor unit located within said interior space in communication with the control unit;
wherein said device receives said wheeled article within said interior space, said sensor detects at least one operating condition and communicates said operating condition to said control unit, and said control unit selectively powers said plurality of UV light sources to emit UV light from said UV light source toward said wheeled article for a defined period of time based upon said at least one operating condition.

13. The device of claim 11, wherein said interior space is defined by at least two side panels and wherein a reflective material is disposed on an interior surface of each of said at least two side panels.

14. The device of claim 11, wherein said at least one UV light source comprises a 222 nanometer UV light lamp.
